(19) United States
(12) Patent Application Publication
(10) Pub. No.: US 2012/0041247 A1
(43) Pub. Date: Feb. 16, 2012

(54) CATALYST FOR PRODUCING ALKENE, THE METHOD FOR PRODUCING THE SAME AND THE METHOD FOR PRODUCING ALKENE

(75) Inventors: Gen Inoue, Oita (JP); Meiko Saihata, Oita (JP)

(73) Assignee: SHOWA DENKO K.K., Minato-ku, Tokyo (JP)

(21) Appl. No.: 13/265,761

(22) PCT Filed: May 27, 2010
(86) PCT No.: PCT/JP2010/059570

§ 371 Date: Oct. 21, 2011

(30) Foreign Application Priority Data
May 27, 2009 (JP) 2009-128120

(51) Int. Cl. C07C 5/48; B01J 27/057

(52) U.S. Cl. 585/658; 502/215

(57) ABSTRACT

The present invention relates to a catalyst for producing alkene using as a starting material alkane obtained by reducing an Mo—V—Te composite oxide having a crystal structure and being represented by formula (1):

MoₐV_{b}Te_{c}O_{d} (1)
wherein a is 1.0, b is from 0.01 to 1.0, c is from 0 to 1.0, and d is the number of oxygen atoms required to electrically neutralize the whole compound determined by the oxidation number of Mo, V and Te; a method for producing the same; and a method for producing alkenes from alkanes using the catalyst.

The catalyst of the present invention enables improvement of the initial activity of the catalyst for producing alkene and selectivity of alkene.

### TECHNICAL FIELD

The present invention relates to a catalyst for producing alkene from alkane. Specifically, the present invention relates to a catalyst for producing alkene using as a starting material alkane obtained by reducing a Mo—V—Te composite oxide having a crystal structure and being represented by formula (1):

MoₐV_{b}Te_{c}O_{d} (1)

wherein a is 1.0, b is from 0.01 to 1.0, c is from 0 to 1.0, and d is the number of oxygen atoms required to electrically neutralize the whole compound determined by the oxidation number of Mo, V and Te; a method for producing the same; and a method for producing alkenes from alkanes using the catalyst.

### BACKGROUND ART

Examples of a method for industrially producing alkens from alkanes include a method for obtaining ethylene from an ethane cracker using ethane as a starting material as described in Petrotech Vol. 28, No. 9, pp. 699-703 (Non-patent Document 1).

A method of obtaining alkenes from alkanes by a catalytic reaction has also been studied. For example, a method for obtaining ethylene from ethane and obtaining propylene from propane by dehydrogenation with a mesoporous zeotype catalyst is disclosed in JP-A-2004-189743 (U.S. Pat. No. 7,045,671; Patent Document 1).

However, since the reaction temperature reaches 500° C. or higher in any of the above methods and the reaction requires a large amount of heat, further technical improvements are being made. An example of the improved methods is oxidative dehydrogenation. Since the oxidative dehydrogenation requires a far smaller amount of heat compared to the method using a cracker and the dehydrogenation method, the oxidative dehydrogenation has potential to become an energy-saving process.

In the oxidative dehydrogenation of alkanes, a composite oxide composed of Mo and V elements (hereinafter referred to as “Mo—V composite oxide”) has been widely used as a catalyst. For example, MoₐVᵥTaₓTe_{y}O_{z} (wherein a is 1.0; v, x and y is from about 0.01 to about 1.0 respectively; and z is the number of oxygen atoms required to neutralize the compound) is used as a catalyst in JP-A-2008-545743 (WO 2006/130288; Patent Document 2). Though many other catalysts have been studied, the reaction temperature reaches 400° C. or higher or pressure exceeding 1 MPa is required under the condition of the reaction temperature around 300° C. in each case.

When severe conditions such as high temperature and high pressure are required, not only a large sum of initial investment is needed for advanced materials of the production equipment, a compressor and the like, but the operations will be complicated. Hence, development of a catalyst capable of being reacted under milder conditions has been demanded.

In recent years, Ueda et al. reported a synthesis of a crystalline oxide of Mo₃V₁Oₓ, which has high activity in the oxidative dehydrogenation reaction of alkanes (e.g. Science and Technology in Catalysis, 91-96, 2006; Non-patent Document 2). Though the document teaches that ethylene and acetic acid can be obtained from ethane under atmospheric pressure at a temperature of from 260 to 360° C., a higher-performance catalyst has been demanded industrially.

### PRIOR ART

### Patent Documents

[Patent Document 1] JP-A-2004-189743 (U.S. Pat. No. 7,045,671)
[Patent Document 2] JP-A-2008-545743 (WO 2006/130288)

### Non-Patent Documents

[Non-patent Document 1] Petrotech Vol. 28, No. 9, pp. 699-703)
[Non-patent Document 2] Science and Technology in Catalysis, 91-96, 2006

### DISCLOSURE OF THE INVENTION

### Problems to be Solved by the Invention

An objective of the present invention is to provide a crystalline MoₐV_{b}Te_{c}O_{d} catalyst having improved activity for producing alkenes by the oxidative dehydrogenation reaction of alkanes.

### Means to Solve the Problem

As a result of intensive studies to solve the above problems, the present inventors found out that a Mo—V-based catalyst obtained by reducing a crystalline Mo—V composite oxide or a crystalline Mo—V—Te composite oxide further containing Te has high activity in the reaction of synthesizing alkenes from alkanes.

That is, the present invention relates to a catalyst for producing alkenes as described in [1] to [3] below, a production method of a catalyst for producing alkenes as described in [4] to [7] below and a method for producing alkenes as described in [8] to [10] below.

[1] A catalyst for producing alkene using as a starting material alkane obtained by reducing an Mo—V—Te composite oxide having a crystal structure and being represented by formula (1):

MoₐV_{b}Te_{c}O_{d} (1)

wherein a is 1.0, b is from 0.01 to 1.0, c is from 0 to 1.0, and d is the number of oxygen atoms required to electrically neutralize the whole compound determined by the oxidation number of Mo, V and Te.
[2] The catalyst for producing alkene as described in [1] above, wherein c is from 0.001 to 0.3 in formula (1).
[3] The catalyst for producing alkene as described in [1] above, wherein c is 0 in formula (1) using as a starting material alkane obtained by reducing a Mo—V composite oxide having a crystal structure and being represented by formula (2), which equals to formula (1) wherein c is 0:

MoₐV_{b}O_{d} (2)

wherein a is 1.0, b is from 0.01 to 1.0, and d is the number of oxygen atoms required to electrically neutralize the whole compound determined by the oxidation number of Mo and V.
[4] A production method of a catalyst for producing alkene using as a starting material alkane, comprising a process for reducing an Mo—V—Te composite oxide (reduction process) having a crystal structure and being represented by formula (1):

MoₐV_{b}Te_{c}O_{d} (1)

wherein a is 1.0, b is from 0.01 to 1.0, c is from 0 to 1.0, and d is the number of oxygen atoms required to electrically neutralize the whole compound determined by the oxidation number of Mo, V and Te.
[5] The production method of a catalyst for producing alkene as described in [4] above, wherein the reducing agent used in the reduction process is one or more members selected from the group of alcohol, hydrogen gas and hydrazine.
[6] The production method of a catalyst for producing alkene as described in [4] or [5] above, comprising a process for calcining a Mo—V—Te composite oxide having a crystal structure (calcination process) represented by formula (1) before the reduction process.
[7] The production method of a catalyst for producing alkene as described in any one of [4] to [6] above, comprising a process for subjecting an Mo—V—Te composite oxide having a crystal structure represented by formula (1) to pressure treatment (pressurization process) before and/or after the reduction process.
[8] A method for producing alkene, comprising heating alkane in the presence of the catalyst for producing alkene as described in any one of [1] to [3] above.
[9] The method for producing alkene as described in [8] above, comprising heating in the presence of oxygen.
[10] The method for producing alkene as described in [8] or [9] above, wherein alkane is ethane and alkene is ethylene.

### Effect of the Invention

The present invention enables to improve the initial activity of a catalyst for producing alkene as well as selectivity of alkene by subjecting a Mo—V composite oxide having a crystalline structure or a Mo—V—Te composite oxide having a crystalline structure to the reduction treatment.

### BRIEF DESCRIPTION OF DRAWINGS

FIG. 1 is an X-ray diffraction profile of the Mo—V composite oxide powder (catalyst 1) in Example 1.

FIG. 2 is an X-ray diffraction profile of the Mo—V—Te composite oxide powder (catalyst 7) in Example 7.

The present invention is hereinafter described in details.

### [Crystalline Mo—V—Te Composite Oxide]

The catalyst for producing alkene of the present invention is represented by formula (1)

MoₐV_{b}Te_{c}O_{d} (1)

wherein a is 1.0, b is from 0.01 to 1.0, c is from 0 to 1.0, and d is the number of oxygen atoms required to electrically neutralize the whole compound determined by the oxidation number of Mo, V and Te; and obtained by reducing an Mo—V—Te composite oxide having a crystalline structure.

The ratio of Mo atoms to V atoms in the Mo—V—Te composite oxide (hereinafter, the composite oxide which does not contain Te: i.e. the case wherein c is 0 in formula (1), is also included in the composite oxide which is referred to as “a crystalline Mo—V—Te composite oxide”) is Mo:V=1.0:0.01 to 1.0, preferably Mo:V=1.0:0.2 to 0.6, more preferably Mo:V=1.0:0.3 to 0.5. When the Mo atom ratio is less than 0.01 or exceeds 1.0, the crystallinity becomes lower and the catalyst performance is lowered. When the V atom ratio exceeds 1.0, alkane is oxidized excessively, leading to reduction in yield of alkene.

The Te atom ratio is Mo:Te=1.0:0 to 1.0, preferably 1.0:0.001 to 0.3. Though the present invention can exert its effect without the presence of the Te atom, the catalyst performance can be further improved when the Te atom is contained in the composite oxide. When the Te atom ratio is within the range of 0.01 to 0.1, the catalyst activity in the oxidation of alkane is improved. The Te atom ratio exceeding 1.0 lowers crystallinity and may degrade the catalyst performance.

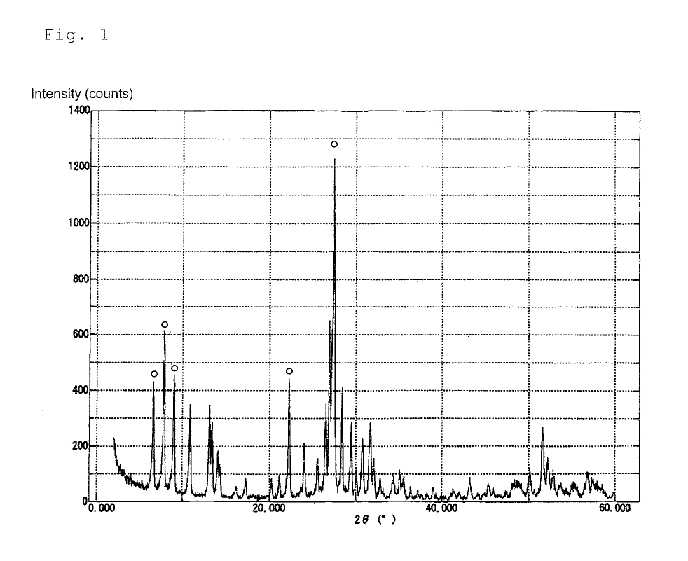

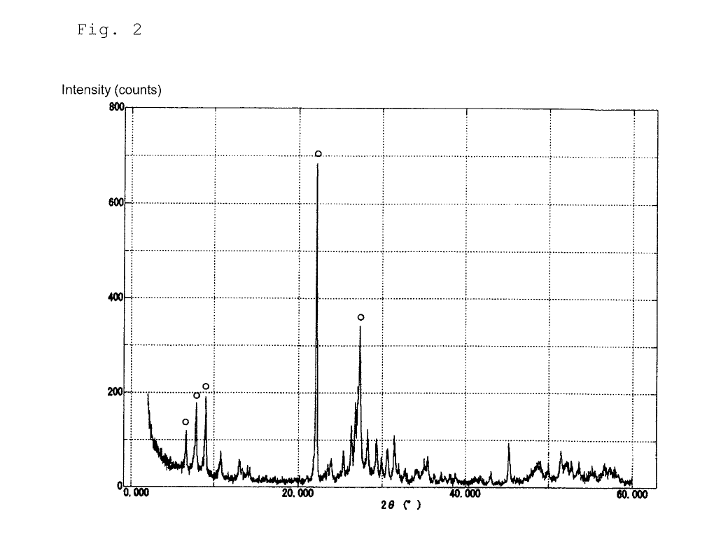

In the present invention, a crystalline composite oxide means a compound, wherein a peak derived from the crystals is observed at the diffraction angle shown in FIG. 1 and FIG. 2 when the X-ray diffraction of the powder is measured. In this case, as long as the presence of the peak derived from crystals is confirmed, the intensity of the peak does not matter.

Accordingly, the crystalline Mo—V composite oxide of the present invention may contain an amorphous structure, but it is preferable that the composite oxide has as high crystallinity as possible. Whether the compound is crystalline or not is determined by the presence of the X-ray diffraction peak at 6.7°, 7.9°, 9.0° 22.2° and 27.3° as a diffraction angle 2θ(±0.3°. It is often the case that such a crystalline compound is a prismatic-shape crystal having a longest size of about 0.01 to 30 μm and a shortest size of about 0.001 to 1 μm which diameters can be observed by a scanning electron microscope.

Examples of the structures of a specifically preferable crystalline compound include those shown in FIG. 1 and FIG. 2 at page 93 of Science and Technology in Catalysis, 2006.

### [Method for Producing a Catalyst]

The catalyst for producing alkene of the present invention can be produced by a method comprising steps 1 to 4 as follows:
1. a process for synthesizing a crystalline Mo—V—Te composite oxide (synthesis process),
2. a process for calcining a crystalline Mo—V—Te composite oxide (calcination process),
3. a process for reducing a crystalline Mo—V—Te composite oxide (reduction process), and
4. a process for subjecting a crystalline Mo—V—Te composite oxide to pressure treatment (pressurization process).

Among the above-mentioned processes, the reduction process in 3 is essential in the present invention.

It is desirable to perform the calcination process in 2 before the reduction process. Although the pressurization process in 4 may be performed either before or after the reduction process, it is preferable to perform after the reduction process. Though the pressurization process may be performed before the calcination process, it is preferable to perform after the calcination process.

Each of the processes is to be described below.

### 1. Synthesis Process

There are no particular limitations on the material compound to be used for the synthesis of the crystalline Mo—V—Te composite oxide of the present invention. Examples of the Mo source (an Mo compound) include ammonium molybdate, molybdenum trioxide, molybdenum acid and sodium molybdate. These compounds are water-soluble and suitable as a material for hydrothermal synthesis. These Mo material compounds may be used singly or in combination of two or more thereof.

There are no particular limitations on the V source (a V compound) either. Examples of the compound include ammonium metavanadate, vanadyl oxalate, vanadium oxide (V₂O₅), ammonium vanadate, vanadyl sulfate, vanadyl nitrate and VO(acac)₂. These compounds are water-soluble and suitable as a material for hydrothermal synthesis. These V material compounds may be used singly or in combination of two or more thereof.

There are no particular limitations on the Te source (Te compound), either. Examples of the compound include telluric acid (H₆TeO₆), TeO₂, potassium tellurate, TeCl₄, Te(OC₂H₅)₅ and Te(OCH(CH₃)₂)₄. Telluric acid and TeO₂ are particularly preferable.

The composition ratio of Mo compound, V compound and Te compound as starting materials is preferably within the range of: molybdenum (Mo):vanadium (V):tellurium (Te) is 1:0.2 to 0.6:0.001 to 0.3, more preferably within the range of Mo:V:Te=1:0.3 to 0.5:0.01 to 0.1 (atom ratio) in the obtained crystalline Mo—V—Te composite oxide. An aqueous mixture of the raw materials is prepared by pouring the compounds, preferably, into water. At this time, it is desirable to disperse the starting materials uniformly in a solvent to be made into a solution or slurry. There are no limitations on the preparation method and the mixing sequence. For example, a method of preparing aqueous solutions each of which contains Mo compound, V compound and Te compound separately in advance, adding the V solution to the Mo solution and subsequently adding thereto the Te solution can be employed. Or the materials may be poured into water all at once thereby to prepare an aqueous mixture of the raw materials. A preferable method is preparing an aqueous solution containing Mo and Te and mixing thereto an aqueous solution of V separately prepared. In the case of preparing an aqueous mixture of raw materials of a high concentration, a method of adding the V solution by drops into the aqueous solution containing Mo and Te is employed so as to maintain the uniformity of the solution. The amount of water to be used should be sufficient as long as it can dissolve the raw materials or make the raw materials into uniform slurry if the water cannot fully dissolve the materials. The amounts of Mo compound, V compound and Te compound are about 0.05 to 50 mol, 0.01 to 1.0 mol and 0 to 0.5 mol, respectively, to 1 liter of water. The pH of the obtained aqueous mixture of the raw materials is to be adjusted to 2 to 4, preferably.

A Mo—V—Te composite oxide may be synthesized by any method which enables synthesis of a crystalline compound. Examples of suitable methods include a method of drying and calcining the above-mentioned aqueous mixture of the raw materials and a hydrothermal synthesis method. The hydrothermal synthesis method is particularly preferable.

The hydrothermal synthesis method is a method of reacting an aqueous material mixture in which an Mo material, V material and Te material as needed are mixed by heating the mixture put into a pressure tight case such as an autoclave. It is preferable to substitute part or all of the air inside the autoclave with inert gas such as nitrogen and helium before conducting the reaction. The oxygen atoms of the Mo—V—Te composite oxide are supplied from the oxygen atoms in water, Mo material, V material and Te material. The presence of oxygen molecules in a gaseous state may reduce the yield of the composite oxide. The reaction temperature of the hydrothermal synthesis is 100 to 400° C., preferably 150 to 250° C. When the reaction temperature is lower than 100° C., the yield of the crystalline composite oxide extremely declines. The reaction temperature may exceed 400° C. but it will increase costs for equipment such as a pressure tight case. The reaction time is generally from one to 100 hours, preferably from 12 to 72 hours. The pressure inside the autoclave is saturated vapor pressure but may vary as needed. Stirring may be carried out during the hydrothermal synthesis.

The reaction solution after the hydrothermal synthesis is completed is cooled and the solid substance contained in the reaction solution is filtrated, washed with water and dried. The drying temperature is not particularly limited and preferably 50 to 200° C., more preferably 110 to 200° C.

The obtained solid substance may be washed with an appropriate solvent. For example, by subjecting the solid substance to heating treatment in an oxalic solution, components having low crystallinity are removed by the oxalic acid and a crystalline Mo—V—Te composite oxide having higher crystallinity can be obtained.

A support can be mixed as needed. Examples of the support include silica, alumina, silica-alumina, titania and zirconia. These supports may be added at the time of synthesizing a crystalline Mo—V—Te composite oxide or may be mixed in a crystalline Mo—V—Te composite oxide after the hydrothermal synthesis thereby to be subjected to calcination and the like.

### 2. Calcination Process

It is desirable to heat and calcine the crystalline Mo—V—Te composite oxide obtained in the synthesis process 1 in the gas phase. The calcination temperature is 250° C. or more, preferably, 300 to 650° C. The calcination time is generally from 5 minutes to 20 hours, preferably from one to six hours. The calcination atmosphere may be in either of air atmosphere or inert gas atmosphere. Preferred is an inert gas atmosphere such as nitrogen gas which does not substantially contain oxygen.

### 3. Reduction Process

The obtained crystalline Mo—V—Te composite oxide is subjected to reduction treatment. The method of reduction treatment may be either of vapor-phase reduction or liquid-phase reduction. Upon the reduction treatment, it is desirable to perform the reduction after pulverizing the crystalline Mo—V—Te composite oxide to increase the surface area.

The liquid-phase reduction may be carried out in either of a non-aqueous system using alcohol and hydrocarbons as a solvent or an aqueous system using water. The crystalline Mo—V—Te composite oxide is preferably in a uniform slurry state during the reduction treatment.

As a reduction agent, carboxylic acid and salts thereof, aldehyde, hydrogen peroxide, sugar, polyvalent phenol, diborane, amine, hydrazine and the like may be used. Examples of carboxylic acid and salts thereof include oxalic acid, potassium oxalate, formic acid, potassium formate and ammonium citrate; and examples of sugar include glucose. A preferred reduction agent is hydrazine, formaldehyde, acetaldehyde, hydroquinone, sodium borohydride, potassium citrate and the like; and the most preferable one is hydrazine and alcohol.

Though there are no particular limitations on the temperature of the liquid-phase reduction, it is desirable to select an appropriate temperature depending on the type of the reduction agent. For example, when alcohol is used, the composite oxide may be refluxed at the boiling point of alcohol. When an aqueous solution of hydrazine is used, the reduction treatment can be carried out at room temperature.

The liquid-phase reduction treatment can be carried out in any embodiment. For example, when the treatment is carried out using alcohol, a crystalline Mo—V—Te composite oxide is put into alcohol and subjected to heat treatment for a specific period of time, if necessary. Specifically, the embodiment may be putting a crystalline Mo—V—Te composite oxide in alcohol and subjecting the mixture to heating or reflux treatment while being stirred.

Though there are no particular limitations on the types of alcohol, one having not very high viscosity is preferable from the viewpoint of treating operations. Examples of preferable alcohol include ethanol, i-propanol, n-butanol, s-butanol and t-butanol.

The reduction treatment time may be adjusted appropriately, and generally the time is preferably from about one hour to 200 hours, more preferably from one hour to 100 hours.

The reduction agent used in the gas-phase reduction can be selected from the group of hydrogen gas, carbon monoxide, alcohol, aldehyde and olefins such as ethylene, propene and isobutene. Preferred is hydrogen gas (H₂). In the gas-phase reduction, inert gas such as helium, argon and nitrogen may be added as a diluent.

There are no particular limitations on the reduction temperature, and the temperature is preferably 100 to 500° C. If the temperature is lower than 100° C., the effect becomes insufficient, and when the temperature exceeds 500° C., it may change the structure of the crystalline Mo—V—Te composite oxide. The reduction at a temperature of from 200 to 400° C. is particularly preferable from the viewpoint of improving performance of the crystalline Mo—V—Te composite oxide.

The gas-phase reduction time can be selected appropriately within the range of from 0.1 to 100 hours, preferably from 0.5 to 100 hours.

There are no particular limitations on the shape of the crystalline Mo—V—Te composite oxide at the time of the gas-phase reduction, and the composite oxide may be in the form of powder, or molded into a pellet or sheet.

The gas-phase reduction may be carried out either by a gas flowing method or a batch method.

In a gas flowing method, for example, a crystalline Mo—V—Te composite oxide is charged in a reaction tube, allowing gas added with a reduction agent to pass through. Though there are no particular limitations on the gas flow conditions, it is desirable to allow the gas to pass through at a rate of about one to 10 nl/h to 1 g of a crystalline Mo—V—Te composite oxide. In this case, either of upflow or downflow can be selected but downflow is preferable when the composite oxide is in the powder form.

The reduction gas may be used as being diluted with inert gas as needed. For example, when the reduction gas is hydrogen gas, it may be mixed with nitrogen gas as inert gas and used.

The mixing ratio of hydrogen gas and nitrogen gas is arbitrary, and the two can be mixed at a ratio of hydrogen gas:nitrogen gas=1:99 to 95:5 (volume ratio). If the hydrogen gas concentration is too high, the catalyst is to be reduced intensely, and if the concentration is too low, the catalyst may not be able to be reduced. The mixing ratio is preferably hydrogen gas:nitrogen gas=5:95 to 25:75 (volume ratio).

### 4. Pressurization Process

Pressure treatment in the pressurization process indicates an operation of applying a given pressure to the crystalline Mo—V—Te composite oxide. Examples of the treatment include the one of compressing powder of the crystalline Mo—V—Te composite oxide using a hydraulic press and the like. Though there are no particular limitations on the pressure to be applied, a pressure of 10 to 5000 MPa is preferable, and a pressure of 100 to 500 MPa is particularly preferable. It is assumed that the aspect ratio becomes lower and surface area increases in the prismatic-shape Mo—V—Te composite oxide crystals due to the pressure treatment, thereby enhancing the catalytic performance.

After compression, the crystalline Mo—V—Te composite oxide may be cut into pellets of about 0.5 to 3 mm to be used as a catalyst. Or, the crystalline Mo—V—Te composite oxide may be pulverized again using a mortar and the like after compression to be used as a catalyst in the powder form.

The pressure treatment may be carried out after synthesizing the crystalline Mo—V—Te composite oxide and either of before or after the reduction.

### [Production of Alkenes]

A method for producing alkenes using a catalyst for synthesizing alkenes of the present invention is described below. It is desirable to carry out the alkene synthesis reaction in the present invention in the gas phase using alkane and oxygen gas as the raw materials for the reaction. Preferred alkane is the one having 2 to 5 carbon atoms. Specifically, ethane and propane are preferable. When the alkane is ethane, the reaction formula is as follows:

C₂H₆+½O₂→CH₂=CH₂+H₂O

Though the alkane and oxygen gas as the raw materials may be used at any ratio as long as an explosion can be avoided, the preferable molar ratio is alkane:oxygen gas=1:0.1 to 5, more preferably 1:0.5 to 1.2.

The raw material gas for the reaction containing alkane and oxygen gas may be used as being diluted with diluent such as nitrogen gas, carbon dioxide (CO₂) and rare gas as needed. When alkane and oxygen gas are used as the raw material for the reaction, the preferable ratio of the raw material and the diluent is raw material:diluent=1:0.05 to 9, more preferably 1:0.1 to 3 (by molar ratio).

In the present invention, it is desirable to allow the raw material gas for the reaction to pass through the catalyst layer at the space velocity (SV) of 10 to 15000 hr⁻¹, particularly 300 to 8000 hr⁻¹. When the space velocity is lower than 10 hr⁻¹, there is a possibility that removal of heat of the reaction would be difficult. When the space velocity is higher than 15000 hr⁻¹, equipment such as a compressor may become too large and unpractical.

Water may be added to the raw material gas for the reaction in an amount of 0.5 to 30 mol %, preferably 1 to 20 mol % for the purpose of improving the alkane conversion rate or preventing generation of side products. It is desirable to adjust the additive amount of water depending on the type of the compound to be produced by the reaction.

Though there are no particular limitations on the material of the reactor, a reactor composed of corrosion-resistant materials (e.g. SUS 316L, zirconia and Hastelloy (registered trade name)) is preferable.

The reaction temperature is 100 to 500° C., preferably 120 to 350° C. When the reaction temperature is lower than 100° C., it might decrease the reaction rate. When the reaction temperature is higher than 500° C., it will incur alteration of the Mo—V—Te composite oxide.

The reaction pressure is 0 to 3 MPaG, preferably 0.1 to 1.5 MPaG, most preferably 0.1 to 1.0 MPaG. When the reaction pressure is lower than 0 MPaG, it might decrease the reaction rate. When the reaction pressure is higher than 3 MPaG, equipment such as a reaction tube will become expensive and unpractical.

Though there are no particular limitations on alkane as a raw material, generally, it is preferable to use high-purity alkane.

There are no particular limitations on the oxygen gas as an oxidizing agent, either. The oxygen gas may be used as being diluted with inert gas such as nitrogen gas and carbon dioxide, for example, may be supplied in the form of air. It is advantageous to use a highly-concentrated oxygen, preferably an oxygen gas having purity of 99% or more, to allow the reaction gas to circulate.

There are not particular limitations on the reaction method, and examples of the method include a known method such as a fixed bed method and a fluid bed method. It is advantageous from a practical view point to employ a fixed bed composed of a corrosion-resistant reaction tube charged with the above-mentioned catalyst.

### EXAMPLES

Hereinafter, the present invention will be explained in more detail with reference to Examples, but the present invention is not limited thereto.

### Example 1

### Catalyst 1

### [Synthesis of Crystalline Mo—V Composite Oxide (A)] (the Composition of the Materials of Mo:V=1:0.25)

(1) (NH₄)₆Mo₇O₂₄.4H₂O (8.82 g, 7.14 mmol, white crystals; manufactured by Wako Pure Chemical Industries, Ltd.) was put into a 200 ml-volume beaker and dissolved in water (120 ml) added thereto (the solution is referred to as “solution 1”).
(2) VOSO₄.nH₂O (3.28 g, n=5.4, 12.5 mmol, blue crystals; manufactured by Mitsuwa Chemicals Co., Ltd.) was put into a 200 ml-volume beaker and dissolved in water (120 ml) added thereto (the solution is referred to as “solution 2”).
(3) The solutions 1 and 2 prepared as described above were transferred to a 300 ml-volume beaker, mixed and stirred with a magnetic stirrer for ten minute to thereby prepare a reaction solution.
(4) The reaction solution prepared in above (3) was poured into an autoclave and N₂ gas was injected into the reaction solution for ten minutes.
(5) The autoclave was sealed and heated at 200° C. for 24 hours.
(6) After cooling to room temperature, the generated purple-black solid in the film state was filtrated and washed with water.
(7) The obtained solid was dried at 80° C. overnight.
(8) The obtained solid was added to a 0.4M aqueous oxalic acid solution (manufactured by Wako Pure Chemical Industries, Ltd.), stirred at 60° C. for 30 minutes, filtrated, washed with water and dried.
(9) The obtained solid was subjected to calcination treatment at 400° C. for two hours under nitrogen stream to thereby obtain a crystalline Mo—V composite oxide (A).

### [Reduction Process]

Crystalline Mo—V composite oxide (A) obtained as described above was heated at 350° C. for two hours under the stream of a mixed gas of hydrogen and nitrogen (H₂:N₂=7:93, 70 ml/min.), thereby carrying out the reduction treatment.

### [Pressurization Process]

3 g of crystalline Mo—V composite oxide (A) after being subjected to reduction treatment was charged in a ring 30 millimeters in inner diameter made from vinyl chloride resin, subjected to pressure treatment at 100 kN for two minutes using BRE-32 manufactured by Maekawa Testing Machine Mfg. Co., LTD., and then pulverized lightly to thereby obtain pellets of a crystalline Mo—V composite oxide (catalyst 1), wherein the pellets are about two millimeters thick.

### Example 2

### Catalyst 2

Crystalline Mo—V composite oxide (A) obtained in the synthesis process of Example 1 was subjected to pressure treatment at 100 kN for two minutes and consequently made into pellets about two millimeters thick. Next, the pellets were heated at 350° C. for two hours under the stream of a mixed gas of hydrogen and nitrogen (H₂:N₂=7:93; 70 ml/min.) to thereby obtain catalyst 2.

### Comparative Example 1

### Comparative Catalyst 1; Heating Treatment in a Nitrogen Atmosphere

Crystalline Mo—V composite oxide (A) obtained in the synthesis process of Example 1 was subjected to heating treatment at 350° C. for two hours in nitrogen stream, and next subjected to pressure treatment at 100 kN for two minutes and consequently made into pellets about two millimeters thick to thereby obtain comparative catalyst 1.

### Comparative Example 2

### Comparative Catalyst 2; Heating Treatment in an Air Atmosphere

Crystalline Mo—V composite oxide (A) obtained in the synthesis process of Example 1 was subjected to heating treatment at 350° C. for two hours in air stream, and next subjected to pressure treatment at 100 kN for two minutes and consequently made into pellets about two millimeters thick to thereby obtain comparative catalyst 2.

### Example 3

### Catalyst 3; Reduction Treatment with i-PrOH

3 g of crystalline Mo—V Composite Oxide (A) obtained in the synthesis process of Example 1 was put into 300 ml of isopropyl alcohol (i-PrOH) and subjected to reduction treatment under reflux at 83° C. for 60 hours. After the treatment, the composite oxide was filtrated and recovered, and thereby to be calcined in a muffle furnace in N₂ atmosphere at 400° C. for two hours. Subsequently, the composite oxide was subjected to pressure treatment at 100 kN for two minutes and made into pellets about two millimeters thick to thereby obtain catalyst 3.

### Example 4

### Catalyst 4; Reduction Treatment Using s-BuOH

Catalyst 4 was obtained in the same manner as in Example 3 except that i-PrOH was substituted with s-butanol (s-BuOH) and the reduction treatment was carried out at 98° C. for 30 hours.

### Example 5

### Catalyst 5; Reduction Treatment Using t-BuOH

Catalyst 5 was obtained in the same manner as in Example 3 except that i-PrOH was substituted with t-butanol (t-BuOH).

### Example 6

### Catalyst6; Reduction Treatment Using Hydrazine

Catalyst 6 was obtained in the same manner as in Example 3 except that i-PrOH was substituted with an aqueous solution of hydrazine (3 mass % of hydrazine monohydrate) and the reduction treatment was carried out at room temperature for seven hours.

### Example 7

### Catalyst 7

### [Synthesis of Crystalline Mo—V—Te Composite Oxide (B)] (the Composition of the Materials of Mo:V:Te=1:0.33:0.023)

(1) (NH₄)₆Mo₇O₂₄.4H₂O (35.3 g, 28.6 mmol, white crystals; manufactured by Wako Pure Chemical Industries, Ltd.) was put into a 500 ml-volume beaker and dissolved in water (320 ml) added thereto (the solution is referred to as “solution 3”).
(2) H₆TeO₆ (1.07 g, 4.67 mmol, white crystals; manufactured by Mitsuwa Chemicals Co., Ltd.) was added to and dissolved in solution 3.
(3) VOSO₄.nH₂O (17.4 g, n=5.4, 66.7 mmol, blue crystals; manufactured by Mitsuwa Chemicals Co., Ltd.) was put into a 500 ml-volume beaker and dissolved in water (320 ml) added thereto (the solution is referred to as “solution 4”).
(4) The solutions 3 and 4 prepared as described above were transferred to a 1 liter-volume beaker, mixed and stirred with a magnetic stirrer for ten minute to thereby prepare mixed liquid 5.
(5) The pH of mixed liquid 5 was adjusted to 3.2 using 25 vol % aqueous ammonia solution.
(6) Mixed liquid 5 prepared in above (5) was poured into an autoclave and N₂ gas was injected into the reaction solution for ten minutes.
(7) The autoclave was sealed and heated at 200° C. for 12 hours.
(8) After cooling to room temperature, the generated purple-black solid in the film state was filtrated and washed with water.
(9) The obtained substance was dried at 80° C. overnight.
(10) The solution was subjected to calcination treatment at 400° C. for two hours under nitrogen stream to thereby obtain a crystalline Mo—V—Te composite oxide (B).

### [Pressure Treatment]

3.5 g of crystalline Mo—V—Te composite oxide (B) was charged in a ring 30 millimeters in inner diameter made from vinyl chloride resin, subjected to pressure treatment at 200 kN for one minute using BRE-32 manufactured by Maekawa Testing Machine Mfg. Co., LTD., and then pulverized lightly to thereby obtain pellets of a crystalline Mo—V—Te composite oxide, wherein the pellets are about two millimeters thick.

### [Reduction Process]

Crystalline Mo—V—Te composite oxide (B) obtained as described above was heated at 300° C. for two hours under the stream of a mixed gas of hydrogen and nitrogen (H₂:N₂=10:90, 100 ml/min.), thereby carrying out the reduction treatment.

### Comparative Example 3

### Comparative Catalyst 3; Preparation of a Catalyst without Reduction Treatment

The pellets of crystalline Mo—V—Te composite oxide after the pressure treatment in Example 7 was used as a catalyst (comparative catalyst 3) as it is without carrying out the reduction treatment.

### Example 8

### Catalyst 8

[Synthesis of crystalline Mo—V—Te composite oxide (C)] (the Composition of the Materials of Mo:V:Te=1:0.33:0.013)

Crystalline Mo—V—Te composite oxide (C) was obtained in the same manner as in Example 7 except that solution 3 was prepared using H₆TeO₆ (0.612 g, 2.67 mmol, white crystals).

The obtained Mo—V—Te composite oxide (C) given above was subjected to pressure treatment at 200 kN for one minute and subsequently made into pellets about two millimeters thick. Next, the pellets were heated at 300° C. for two hours under the stream of a mixed gas of hydrogen and nitrogen (H₂:N₂=10:90, 100 ml/min.), thereby carrying out the reduction treatment.

### Comparative Example 4

### Comparative Catalyst 4; Preparation of a Catalyst without Reduction Treatment

The pellets obtained by subjecting crystalline Mo—V—Te composite oxide (C) in Example 8 to the pressure treatment was used as a catalyst (comparative catalyst 4) as it is without carrying out the reduction treatment.

[Composition Analysis] 0.5 g of each of catalysts 1 to 8 prepared in Examples 1 to 8 was charged in a ring 10 millimeters in inner diameter made from vinyl chloride resin and subjected to pressure treatment at 50 kN for one minute using BRE-32 manufactured by Maekawa Testing Machine Mfg. Co., LTD. Obtained disks were subjected to fluorescent X-ray analysis (XRF) using ZSX primusII manufactured by Rigaku Corporation. The analysis results are shown in Table 1.

TABLE 1

| | | | Compositions | | |
|---|---|---|---|---|---|
| | | Catalyst | (molar ratio) | | |
| Examples | | No. | Mo | V | Te |
| Example 1 | Mo—V composite oxide (A) | 1 | 1.0 | 0.4 | 0.00 |
| Example 2 | Mo—V composite oxide (A) | 2 | 1.0 | 0.4 | 0.00 |
| Example 3 | Mo—V composite oxide (A) | 3 | 1.0 | 0.4 | 0.00 |
| Example 4 | Mo—V composite oxide (A) | 4 | 1.0 | 0.4 | 0.00 |
| Example 5 | Mo—V composite oxide (A) | 5 | 1.0 | 0.3 | 0.00 |
| Example 6 | Mo—V composite oxide (A) | 6 | 1.0 | 0.4 | 0.00 |
| Example 7 | Mo—V—Te composite oxide (B) | 7 | 1.0 | 0.5 | 0.04 |
| Example 8 | Mo—V—Te composite oxide (C) | 8 | 1.0 | 0.4 | 0.02 |

### [XRD Measurement]

After crystalline Mo—V composite oxide catalyst (catalyst 1) in the form of pellets prepared in Example 1 or crystalline Mo—V—Te composite oxide catalyst (catalyst 7) obtained in Example 7 was pounded in a mortar, the catalyst was put into a sample holder and pressed by a glass plate to level the surface, thereby being subjected to X-ray diffraction measurement using MultiFlex manufactured by Rigaku Corporation. The measurement results are shown in FIG. 1 and FIG. 2.

As the peaks showing that the catalyst is crystalline (2θ(±0.3°)), angles of 6.7°, 7.9°, 9.0°, 22.2° and 27.3° were marked with o.

### Measurement Conditions:

X-ray source: CuKα, output: 50 kV, current: 20 mA, measurement range (2θ): 5 to 60°, scan method: continuous measurement, STEP: 0.01°, time/step: 0.5 sec.

### [Reaction Evaluation Test 1]

2 g each of the catalysts prepared in Examples 1 to 7 and Comparative Examples 1 to 3 was charged into a reaction tube (size: inner diameter of 5 mm, length of 200 mm, material: SUS316L) and subjected to a reaction with a material gas having compositions of C₂H₆:O₂:H₂O:N₂=10:10:10:70 (by molar ratio) at a gas flow rate of 6.0 nl/h (space velocity (SV)=3000 hr⁻¹), a reaction temperature of 280° C. and reaction pressure of atmospheric pressure. The outlet gas of the reactor was analyzed by a method as described below.

### 1. Oxygen, Nitrogen and CO

50 ml of the outlet gas was sampled and the total gas was introduced into a 1 ml-volume gas sampler attached to the gas chromatography to be analyzed by the absolute calibration method under the following conditions.

Gas chromatography: gas chromatography (GC-14(B); manufactured by SHIMADZU Corporation) provided with SHIMADZU gas sampler for gas chromatograph (MGS-4; 1 ml-volume measuring tube)

Column: MS-5A IS 60/80 mesh (3 mmΦ)×3 m)

Carrier gas: helium (flow rate: 20 ml/min.)

Temperature conditions: 110° C. in the detector and in the vaporizing chamber and 70° C. in a column (constant)

Detector: TCD (He pressure: 70 kPaG, current: 100 m (A))

### 2. Ethane, Ethylene and CO₂

50 ml of the outlet gas was sampled and the total gas was introduced into a 1 ml-volume gas sampler attached to the gas chromatography to be analyzed by the absolute calibration method under the following conditions.

Gas chromatography: gas chromatography (GC-14(B); manufactured by SHIMADZU Corporation) provided with SHIMADZU gas sampler for gas chromatograph (MGS-4; 1 ml-volume measuring tube)

Column: Unibeads IS 60/80 mesh (3 mmΦ×3 m)

Carrier gas: helium (flow rate: 20 ml/min.)

Temperature conditions: 100° C. in the detector and in the vaporizing chamber and 60° C. in a column (constant)

Detector: TCD (He pressure: 70 kPaG, current: 100 m (A))

### 3. Acetic Acid

10 ml of reaction solution with 1 ml of 1,4-dioxane was added thereto as an internal standard was taken as a sample solution and 0.2 μl of the solution was introduced to be analyzed by the internal standard method under the following conditions.

gas chromatography: GC-14B manufactured by SHIMADZU Corporation

column: packed column, Thermon 3000 (length of 3 m and inner diameter of 0.3 mm)

carrier gas: nitrogen (flow rate: 20 ml/min.)

temperature conditions: 180° C. in the detector and in the vaporizing chamber; the column temperature was maintained at 50° C. for the first six minutes after the analysis started and heated to 150° C. at a heating rate of 10° C./min. and maintained at 150° C. for ten minutes.

detector: FID (H₂ pressure: 40 kPaG, air pressure: 100 kPaG)

### [Reaction Evaluation Test 2]

The test was carried out in the same manner as in Reaction evaluation test 1 except that each 1 g of the catalysts prepared in Example 8 and Comparative Example 4 was used at a space velocity (SV) of 6000 hr⁻¹.

The evaluation results of the initial activity of each of the catalysts in Examples 1 to 6 and Comparative Examples 1 to 2 are shown in Table 2. The specific conversion and specific STY in the table was determined by the following formulae.

Specific conversion=conversion of each example(or comparative example)/conversion of BLANK

Specific STY=STY of each example(or comparative example)/STY of BLANK

TABLE 2

| | | | (first row) | (upper row) STY (g/h · Lcat) | | | |
|---|---|---|---|---|---|---|---|
| | | | Ethane conversion (%) | (second row) specific STY | | | |
| | Mo—V composite | Catalyst | (second row) | | | | Acetic |
| Examples | oxide (A) | No. | Specific conversion (%) | Ethylene | CO | CO₂ | acid |
| BLANK | Pressure treatment only | | 19.0 | 54.0 | 12.0 | 20.0 | 13.0 |
| | | | 1.0 | 1.0 | 1.0 | 1.0 | 1.0 |
| Example | Reduction with H₂ → | 1 | 20.9 | 64.8 | 12.0 | 18.0 | 16.9 |
| 1 | pressure treatment | | 1.1 | 1.2 | 1.0 | 0.9 | 1.3 |
| Example | Pressure treatment → | 2 | 20.9 | 64.8 | 8.4 | 22.0 | 22.1 |
| 2 | reduction with H₂ | | 1.1 | 1.2 | 0.7 | 1.1 | 1.7 |
| Example | Reduction with i-PrOH | 3 | 26.6 | 81.0 | 14.4 | 12.0 | 29.9 |
| 3 | → pressure treatment | | 1.4 | 1.5 | 1.2 | 0.6 | 2.3 |
| Example | Reduction with s-BuOH | 4 | 22.8 | 64.8 | 15.6 | 20.0 | 16.9 |
| 4 | → pressure treatment | | 1.2 | 1.2 | 1.3 | 1.0 | 1.3 |
| Example | Reduction with t-BuOH | 5 | 32.3 | 81.0 | 28.8 | 42.0 | 15.6 |
| 5 | → pressure treatment | | 1.7 | 1.5 | 2.4 | 2.1 | 1.2 |
| Example | Reduction with hydrazine | 6 | 32.3 | 81.0 | 16.8 | 28.0 | 18.2 |
| 6 | → pressure treatment | | 1.7 | 1.5 | 1.4 | 1.4 | 1.4 |
| Compara- | Heating in N₂ → | Compara- | 11.4 | 32.4 | 3.6 | 10.0 | 9.1 |
| tive Ex. 1 | pressure treatment | tive 1 | 0.6 | 0.6 | 0.3 | 0.5 | 0.7 |
| Compara- | Heating in air → | Compara- | 7.6 | 27.0 | 3.6 | 6.0 | 5.2 |
| tive Ex. 2 | pressure treatment | tive 2 | 0.4 | 0.5 | 0.3 | 0.3 | 0.4 |

In each case of the catalysts in Examples subjected to the reduction treatment, the ethane conversion was improved compared to the “BLANK” case and STY (space time yield) was also improved with respect to ethylene and acetic acid. On the other hand, in Comparative Examples in which the catalyst was only heated in air and in nitrogen (without carrying out the reduction treatment), all of the ethane conversion, STY of ethylene and STY of acetic acid showed decrease.

The evaluation results of the initial activity of each of the catalysts in Examples 7 to 8 and Comparative Examples 3 to 4 are shown in Table 3.

TABLE 3

| | | Ethane | STY (g/h · Lcat) | | | |
|---|---|---|---|---|---|---|
| | Catalyst | conversion | | | | Acetic |
| Examples | No. | (%) | ethylene | CO | CO₂ | acid |

Mo—V—Te composite oxide (B)

| | | | | | | | |
|---|---|---|---|---|---|---|---|
| Example 7 | Pressure treatment→ | 7 | 42 | 122 | 33 | 18 | 28 |
| | reduction with H₂ | | | | | | |
| Compara- | Pressure treatment only | Compara- | 42 | 115 | 41 | 23 | 33 |
| tive Ex. 3 | | tive 3 | | | | | |

Mo—V—Te composite oxide (C)

| | | | | | | | |
|---|---|---|---|---|---|---|---|
| Example 8* | Pressure treatment→ | 8 | 16 | 101 | 17 | 12 | 17 |
| | reduction with H₂ | | | | | | |
| Compara- | Pressure treatment only | Compara- | 14 | 88 | 15 | 11 | 15 |
| tive Ex. 4* | | tive 4 | | | | | |

*space velocity (SV) = 6000 hr⁻¹ (SV = 3000 hr⁻¹ in the other examples)

Comparison between Table 2 and Table 3 shows that ethane conversion is improved in crystalline Mo—V—Te composite oxide (B) compared to Mo—V composite oxide (A) to which Te is not added.

When crystalline Mo—V—Te composite oxide (B) was subjected to reduction treatment, ethane conversion remained unchanged but STY (space time yield) of ethylene was improved. Also, when crystalline Mo—V—Te composite oxide (C) was subjected to reduction treatment, ethane conversion was improved as well as the STY of ethylene and STY of acetic acid.

1. A catalyst for producing alkene by the oxidative dehydrogenation reaction of alkane, wherein the catalyst is obtained by reducing an Mo—V—Te composite oxide having a crystal structure and being represented by formula (1):
MoₐV_{b}Te_{c}O_{d} (1)
wherein a is 1.0, b is from 0.01 to 1.0, c is from 0 to 1.0, and d is the number of oxygen atoms required to electrically neutralize the whole compound determined by the oxidation number of Mo, V and Te.

2. The catalyst for producing alkene as claimed in claim 1, wherein c is from 0.001 to 0.3 in formula (1).

3. The catalyst for producing alkene as claimed in claim 1, wherein c is 0 in formula (1) obtained by reducing a Mo—V—Te composite oxide having a crystal structure and being represented by formula (2), which equals to formula (1) wherein c is 0:
MoₐV_{b}O_{d} (2)
wherein a is 1.0, b is from 0.01 to 1.0, and d is the number of oxygen atoms required to electrically neutralize the whole compound determined by the oxidation number of Mo and V.

4. A production method of a catalyst for producing alkene using as a starting material alkane, comprising a process for reducing an Mo—V—Te composite oxide (reduction process) having a crystal structure and being represented by formula (1):
MoₐV_{b}Te_{c}O_{d} (1)
wherein a is 1.0, b is from 0.01 to 1.0, c is from 0 to 1.0, and d is the number of oxygen atoms required to electrically neutralize the whole compound determined by the oxidation number of Mo, V and Te.

5. The production method of a catalyst for producing alkene as claimed in claim 4, wherein the reducing agent used in the reduction process is one or more members selected from the group of alcohol, hydrogen gas and hydrazine.

6. The production method of a catalyst for producing alkene as claimed in claim 4, comprising a process for calcining a Mo—V—Te composite oxide having a crystal structure (calcination process) represented by formula (1) before the reduction process.

7. The production method of a catalyst for producing alkene as claimed in claim 4, comprising a process for subjecting an Mo—V—Te composite oxide having a crystal structure represented by formula (1) to pressure treatment (pressurization process) before and/or after the reduction process.

8. A method for producing alkene, comprising heating alkane in the presence of the catalyst for producing alkene as claimed in claim 1.

9. The method for producing alkene as claimed in claim 8, comprising heating in the presence of oxygen.

10. The method for producing alkene as claimed in claim 8, wherein alkane is ethane and alkene is ethylene.
